(19) United States
(12) Patent Application Publication
(10) Pub. No.: US 2005/0196345 A1
(43) Pub. Date: Sep. 8, 2005

(54) Compressed air inhaler for pulmonary application of liposomal powder aerosols and powder aerosols

(75) Inventors: Julia Eva Diederichs, Berlin (DE); Wolfgang Koch, Steimbke (DE); Hubert Lodding, Lehrte (DE); Regina Reszka, Schwanebeck (DE); Horst Windt, Burgwedel (DE)

Correspondence Address: NORRIS, MCLAUGHLIN & MARCUS, P.A., 875 THIRD AVE, 18TH FLOOR, NEW YORK, NY, 10022, US

(73) Assignees: Max-Delbruck-Centrum fur Molekulare Medizin, Berlin (DE); Fraunhofer-Gesellschaft zur Forderung der Angewandten Forschung E.V., Munchen (DE)

(21) Appl. No.: 11/111,589

(22) Filed: Apr. 21, 2005

Related U.S. Application Data
(62) Division of application No. 09/890,667, filed on Oct. 10, 2001, now abandoned.

(30) Foreign Application Priority Data
Feb. 3, 1999 (DE) 199 05 285.9
Nov. 2, 1999 (DE) 199 54 107.8

(51) Int. Cl. A61L 9/04; A61M 11/00

(52) U.S. Cl. 424/45; 128/200.23

(57) ABSTRACT

The invention relates to a compressed air inhaler for the pulmonary application of a liposomal powder aerosol that carries active agents or is uncharged.

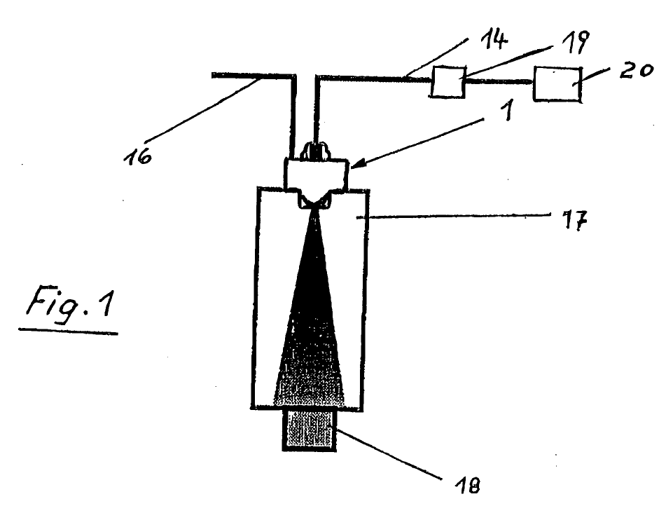

The aim of the invention, providing an inhaler with which the pulmonary application of, in particular, liposomes carrying active agent is enabled without forced breathing manoeuvres and with which the active agent can be transferred to the desired place of action in a sufficient amount without the active agent escaping, is solved by the compressed air inhaler for the pulmonary application of a liposomal powder aerosol entailing a receptacle 20 for a watery liposome dispersion, in which the liposomes carrying the active agent are dispersed in water, which is connected via a liquid dosing device 19 with an atomiser nozzle 1 and with a drying unit 17 such as an atomising chamber for the spray drying of the liposomes, to which said drying unit an exit 18 such as a mouthpiece is connected, in which the atomiser nozzle 1 has separate supply channels 14,16 for the compressed air and the liposome dispersion-FIG. 1-. The aim of the invention is further a new kind of powder aerosol comprising liposomes or nano-particles.

The invention relates to a compressed air inhaler for the pulmonary application of a liposomal powder aerosol that carries active agents or is uncharged.

The invention is preferably intended for use on liposomal powder aerosols carrying active agents. However, the invention can also be used for uncharged liposomes, for example liposomes of specific lipids such as surfactant lipids, which can represent active agents themselves.

Further, the object of the invention is the powder aerosols which carry active agents or are uncharged and which can be used in the compressed air inhaler.

Liposomes are increasingly gaining importance as an application form for active agents. In order to improve the physical stability and the service life of liposomes and to keep the encapsulated active agents, liposome formulations carrying active agents are lyophilised. However, these liposome lyophilisates are hygroscopic and thus cause difficulties in the application if they are to be administered as dry powder. Storage of liposome lyophilisates is always problematic if it cannot be done in a closed vessel in a vacuum. For application, the lyophilisates are reconstructed in water or a normal saline solution and, for example, administered as an intravenous injection. It is also possible to encapsulate the liposome lyophilisates or to press them into a tablet form, in order to provide oral forms of administration.

Liposomes carrying active agents can also be used for treatment of broncho-pulmonary diseases such as asthma, bronchitis or lung cancer.

The results achieved up to now in the use of liposomes for enrichment of medicines in the lung are highly promising. As a result of the liposome-mediated pulmonary application of medicines, the retention time of the active agent in the lung is extended, the therapeutic efficiency being increased with a distinct reduction of the extra-pulmonary side-effects. The extended retention time of the liposomes in the respiratory tract noticeably influences the pharmaco-kinetics of the encapsulated material. There are increased local concentrations with a reduced level of active agents outside the lung. Toxicity examinations show no histological alterations of the lung in vivo and also no toxic effects on healthy candidates in chronic inhalation, which demonstrates a good compatibility of the pulmonary application of liposomes.

For example, U.S. Pat. No. 4,895,719 describes dry liposome powders produced by spray drying, which are suspended in fluorinated hydrocarbons, simultaneously acting as a propellant, and are atomised and inhaled under pressure. The propellant evaporates in the appliance and in the oral cavity. As a result of the breathing process, the air in the appliance and the oral cavity is water-vapour saturated and the propellant is immediately replaced by water as a result of the hygroscopicity of the formulation, with the result that a watery aerosol is inhaled in this case.

In EP 0 223 831 B1, a system for application of a water-soluble medicine in the respiratory tract is described, using liposomes for the encapsulation of the medicine and providing an appliance not described in more detail for the atomisation of a defined quantity of liposomes by means of ultrasound or pneumatically for inhalation.

The generation of liposomal aerosol formulations is not free of problems. For example, in the use of an ultrasonic atomiser, there is a progressive rise in the temperature of the aerosol in the so-called “holding chamber”. This rise in temperature results in a release of the encapsulated material as a function of the phase-transition temperature of the lipids used. In studies in which the liposomal aerosol is generated with the help of medicinal compressed air atomisers, a release of the active agents has also been observed. This phenomenon is put down to the shearing forces during the atomising. This effect is particularly marked in customary medicinal compressed air atomisers, as the liposome suspension to be released as an aerosol for inhalation has to pass through a number of atomisation cycles, as only a small amount which can be inhaled results in the formation of the drips and is produced. The remainder is precipitated in the appliance and used for a further atomisation.

In the customary atomiser, the energy yield is not very effective, which results in a great amount of large drops which cannot be inhaled. These drops flow back into the reservoir. On average, the liposome dispersion circulates more than ten times and is dispersed again each time before leaving the atomiser as an aerosol which can be inhaled. The repeated dispersion process can cause stability problems, in particular for substances which are susceptible to mechanical loads. During the atomisation process, a considerable quantity of water evaporates out of the liposome dispersion, as the atomisation air is dry and the aerosol drops favour the evaporation with a large surface. Therefore, the liposome concentration increases in the course of the atomisation time and thus also the inhaled dose. Therefore, uniformity of the dose is difficult to achieve for liposomes in a customary atomiser. The efficiency of the inhaled therapeutic aerosol depends on various factors, mainly connected with the particle size of the aerosol. Atomisers customarily available generate an average drop size of 5 μm. Such an aerosol is deposited in the upper respiratory tract. But as numerous broncho-pulmonary diseases manifest themselves in lower areas of the lungs, it would be desirable to be able to adapt the range of particle sizes.

Dry powder inhalers (dpi) are also known, demanding the inhalation of dry liposome powders by deep breathing in. Such a dry powder inhaler is portrayed in U.S. Pat. No. 4,895,719 (FIG. 6). This form of application has the disadvantage that patients suffering from diseases of the respiratory tract must be capable of forced breathing manoeuvres in order to breathe the powder in. Also, due to the hygroscopicity of the dry liposomes, there is the risk of the aggregation of the liposome powder and possibly the release of the active agent, through which precise dosage is no longer possible. Due to the aggregation of the liposome powder, the particle size and thus the deposition pattern of the liposomes increase. The areas of the lung in which the effect is to take place are no longer reached. The consequence is an insufficient dosage and effect.

The aim of the invention was thus to provide an inhaler by means of which the pulmonary application of liposomes is possible without forced breathing manoeuvres and with which the active agent can be transferred to the desired place of action in a sufficient amount without the active agent escaping.

A further aim of the invention entailed providing suitable aerosol formulations for application in this inhaler.

The aim of the invention is solved by a compressed air inhaler pursuant to the features of claim 1. Accordingly, the compressed air inhaler for the pulmonary application of a liposome powder aerosol carrying an active agent or also uncharged entails a receptacle for a watery liposome dispersion, in which the liposomes are dispersed in water and which is connected via a pump with an atomiser nozzle and with a drying unit such as an atomising chamber for spray drying the liposomes. A mouthpiece is connected to said drying unit, the atomising nozzle being provided with separate supply channels for the compressed air and the dispersion of the liposomes.

The compressed air inhaler in the invention permits in vivo generation of a powder aerosol on the basis of a watery liposome dispersion, with the result on the one hand that no forced breathing manoeuvres by the patient are necessary and on the other hand no problems arise in the handling of the dry liposome powder. The watery liposome dispersion has low toxicity and a high stability of the liposomes in comparison with organic media and is easy to manufacture with the possibility of scaling up.

It is a benefit that no separate propellant is needed according to the invention and that the size of the aerosol powder particles can be regulated according to the individual situation.

Unlike the known inhalers, there is no multiple atomisation. Everything sprayed is also dried to powder in situ and inhaled immediately, the liposomes do not circulate in the inhaler. The formulation is not subjected to any increased stress.

Purposeful embodiments of the invention are described in the sub-claims.

The invention is described below in an example of an embodiment of a compressed air inhaler on the basis of a figure. The figures show:

FIG. 1 a schematic portrayal of a compressed air inhaler according to the invention and

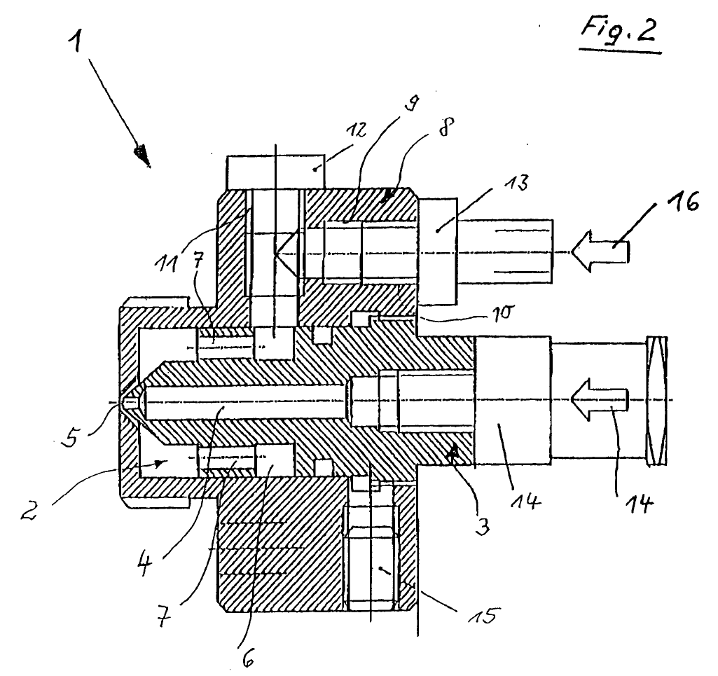

FIG. 2 the schematic sectional portrayal of a nozzle body in the compressed air inhaler according to FIG. 1.

Corresponding to the portrayal in FIG. 1, the compressed air inhaler essentially comprises a nozzle body 1 with a compressed air supply channel 16 and a liquid supply channel 14, a compensatory and drying chamber (atomisation chamber) 17 and an aerosol exit (mouthpiece) 18. The liquid supply channel 14 is connected with a liquid dosage device 19 and a receptacle 20 for the watery liposome dispersion.

To regulate the pressure, the supply channel 16 is connected with a pressure reducer, not portrayed here.

The nozzle body 1 forms a two-material nozzle, in which the supplied liquid is atomised with the help of the supplied compressed air, the pressure of which can be regulated. As a rule, the active agent is dissolved in the supplied liquid, for example water or ethanol, and in the present case is provided as a dispersion (liposomes). The air throughflow and the liquid throughflow can be adjusted independent of one another.

According to the portrayal in FIG. 2, the nozzle body 1 essentially comprises a central cavity 2, into which an injection body 3 is inserted concentrically, having a central bore 4 to supply the liquid and spaced adjustably from a release opening 5 of the nozzle body 1 via a fixing screw 15 and provided with a ring-shaped bore 6 with release openings 7 into the cavity 2 of the nozzle body 1.

In one of the sides 8 of the nozzle body 1, a supply channel 9 for the compressed air is inserted parallel to the walls 10, impacting on a bore 11 vertical thereto, which is connected with the ring-shaped bore 6 of the injection body 2. The bore 11 is closed to the outside by means of a plug 12. The compressed air is fed into the compressed air supply channel 16, for example, by a hose olive 13.

The drop size at the entrance to the drying unit 17 can be set by variation of the pressure of the supplied air and by selecting the diameter of the liquid capillaries (release opening 5 in FIG. 2). If the compressed air is set low, for example p=0.8 bar, mean drop diameters of 8 μm result in the drying unit 17, with a set pressure of p=5 bar, mean drop diameters of 4 μm result.

In addition, the generated drops can be separated from the atomising air before entering the drying unit 17 by the integration of a virtual impactor in a further variant.

As a result of the evaporation in the drying unit 17 of the supplied liquid, which as a rule is water or ethanol and acts as a solvent for the active agent or as a dispersion medium for the liposomes, the atomised drops are reduced in their diameter right down to complete drying. The connection between the size of the dry particle at the exit of the drying unit 17 according to FIG. 1, i.e. at the aerosol exit 18, is stated in the following relation:
dₜᵣ=c^{1/3}dₚᵣᵢₘ,
with
dₜᵣ=diameter of the dry particle at the aerosol exit 18,
dₚᵣᵢₘ=diameter of the pertinent primary drop at the entrance to the drying unit 17,
c=mass concentration of the active agent (dispersion concentration of the liposomes in the watery solution).

In a one per cent solution, there is thus a diameter reduction by a factor of 4.6.

By alteration of the pressure of the supplied air, particles with a mean size of 1.7 μm for example or, with the pressure set higher, 0.9 μm for example can leave the exit of the drying unit 17 of the inhaler (aerosol exit 18, for example a mouthpiece).

Drying unit 17 (or evaporation unit) is dimensioned in such a way that a complete evaporation is guaranteed without considerable losses of drops on the walls.

In order to achieve complete evaporation and thus drying, a certain ratio between the liquid throughflow and the air throughflow must be set, this being determined by the absorption capacity of the compressed air for the liquid (for example water or ethanol vapour).

In a preferred embodiment, the drying unit 17 is equipped with semi-permeable membranes for an increased liquid throughflow for the drying of the aerosol. On the outside of this/these membrane(s), there is, if need be, a drying agent for the absorption of the water vapour. In another variant, the membrane is scavenged with dry air on the outside.

An optimal, high concentration of active agent aerosol is achieved by the adjustment of the flow of liquid and compressed air adjusted separately from one other.

The liquid is only subjected to the dispersion process once. A recirculation of the dispersion and the enrichment of the concentration connected therewith or structural alteration of the active agent particles by repeated effect of high shearing forces is avoided. The drops are dried immediately after being generated.

The compressed air inhaler is planned as a pocket device for easy use by the patient anywhere. In this, the drying unit 17 can be designed very small in its function as a drying element, for example as a hose.

The aim of the invention is also the method for treatment of pulmonary diseases by the compressed air inhaler according to the invention being used.

The aim of the invention is further liposomal and nano-particular powder aerosols according to Claims 7-27.

These powder aerosols have the following properties: it is a dry powder without the presence of a cyro-protector. The individual powder particles are spherical and have an amorphous or crystalline structure. Their size is variable from 0.5-10 μm. In this way, the place of deposition can be set to a certain depth of the lung matching the disease to be treated. They comprise dry, loose liposome or nano-particle aggregates or individual liposomes or nano-particles.

The composition of the powder aerosols can vary in broad ranges. Liposomal powder aerosol comprises, for example, not only the active agent, but also phospholipids and cholesterol in changing ratios, natural or artificial lung surfactant or cationic amphiphiles. The liposomes used can, for example, be large multi-lamellar vesicles (MLV), produced inter alia by homogenisation with and without high pressure, or small uni-lamellar vesicles (SUV), produced inter alia by sonication. Nano-particular powder aerosol comprises, for example, active agent crystals or polymer particles charged with active agents. Inter alia, the following can be used as polymers: polymethacrylate, polycyanoacrylate, polyglycolate, polylactate, polyvinylpyrolidon, polyvinyl acetate, alginate, gelatine individually or in variable mixture ratios. The liposomes or nano-particles can be surface-modified, e.g. with polyethylene glycol, plasma or surfactant-associated proteins or with antibody fragments.

Charged with the corresponding active agent, the invention can be used to treat all known diseases of the respiratory tract. All conventional medications and genetic material can be regarded as active agents. Liposomal powder aerosol without active agent can be used as a substitution therapy of lung surfactant, e.g. in the treatment of shortness of breath syndrome in new-born babies or shock lung.

The liposomal and nano-particular powder aerosols are manufactured by the liposomes or the nano-particular dispersion being atomised together with compressed air via a two-material nozzle in a cylinder (as described above).

The mean aerosol particle size can be adjusted by variation of the dispersion concentration and the primary drop spectrum, for example via the atomisation pressure. In particular, the diameter can be set to match the requirements in the application on a test animal or in man. In this way, the inhaled medication can be deposited in a reproducible quantity, homogeneously and with high efficiency. If a systemic effect is to be achieved in human application, it is an advantage to set the mean particle size to a value of about 1 μm, in order to achieve an optimal particle deposition in the alveolar area of the lung and thus to make use of the large surface of the gas-epithelium border area for absorption of the active agent. For applications for local therapy of bronchial diseases, it is more of an advantage if the particles are deposited in the peripheral lung area, for which particles in the size range of between 3 and 5 μm are optimal.

Particle sizes of about 1 μm result if an atomisation pressure of 3 bar and a dispersion concentration of 0.5% are used. In lower atomisation pressures and increased dispersion concentrations, for example by increasing the liposome concentration or by additives (lactose), the mean particle size can be moved towards larger figures.

The invention provides a method with which the inhaled medication can be reproduced quantitatively and deposited homogeneously.

It is a matter of course and part of the scope of the invention that the powder aerosols described can also be used independent of the inhaler according to the invention.

Own Results:

Up to now, liposomal powder aerosol with the following composition has been successfully produced:
1. Hydrated soy phosphatidylcholin and cholesterol in a molar ratio of 1:0.25
   SUV's produced with ultra-sonication
   Mean particle size of the liposomes: 121 nm
   Mean particle size of the aerosol: 1.02 μm
   Hydrated soy phosphatidylcholin and cholesterol in a molar ratio of 1:0.25
   MLV's produced by homogenisation
   Mean particle size of the liposomes: 1.5 μm
   Mean particle size of the aerosol: 1.03 μm
2. Hydrated soy phosphatidylcholin, cholesterol and polyethylene glycol in a molar ratio of 1:1:0.1
   MLV's produced by shaking the dispersed lipid film
   Mean particle size of the liposomes: 15 μm
   Mean particle size of the aerosol: 0.7 μm
3. Hydrated soy phosphatidylcholin, cholesterol and polyethylene glycol in a molar ratio of 1:1:0.1
   SUV's produced with ultra-sonication
   Mean particle size of the liposomes: 100 nm
   Mean particle size of the aerosol: 0.8 μm
4. Phosphatidylcholin and cholesterol in a molar ratio of 1:0.5 charged with 5.6-carboxyfluorescein
   SUV's produced with ultra-sonication
   Mean particle size of the liposomes: 80 nm
   Mean particle size of the aerosol: 0.8 μm
5. DAC-Chol and DOPE in a weight ratio of 2:3
   Mean particle size of the aerosol: 0.6 μm
6. DAC-Chol and DOPE in a weight ratio of 2:3 complexed with protamine sulphate
   Mean particle size of the aerosol: 0.7 μm
7. DAC-Chol and DOPE in a weight ratio of 2:3 complexed with Poly-L-Lysin Mean particle size of the aerosol: 0.6 μm

Recipe no. 5 was used in an in vivo test for atomisation of 3 BDF1 mice. The test animals were treated for a period of 60 minutes. The aerosol was tolerated well. All told, 385 ng/l of carboxyfluorescein were atomised in the test. The concentration in the individual organs of the test animals is listed below:

| | | Organ weight | CF content in |
|---|---|---|---|
| Mouse no. | Organs | in mg | ng/mg tissue |
| 1 | Trachea | 4.6 | 0.027 |
| | Right lobe of the lung | 35.18 | 0.095 |
| | Left lobe of the lung | 33.55 | 0.074 |
| 2 | Trachea | 16.58 | 0.068 |
| | Right lobe of the lung | 45.68 | 0.068 |
| | Left lobe of the lung | 66.59 | 0.039 |
| 3 | Trachea | 2.53 | 0.072 |
| | Right lobe of the lung | 40.14 | 0.085 |
| | Left lobe of the lung | 65.91 | 0.032 |

List of reference signs

| | |
|---|---|
| 1 | Nozzle body |
| 2 | Cavity |
| 3 | Injection body |
| 4 | Bore |
| 5 | Release opening |
| 6 | Ring-shaped bore |
| 7 | Release opening |
| 8 | Side of 1 |
| 9 | Supply channels |
| 10 | Wall |
| 11 | Vertical bore |
| 12 | Plug |
| 13 | Hose olive |
| 14 | Liquid connection |
| 15 | Fixing screw |
| 16 | Compressed air supply |
| 17 | Drying unit (atomisation chamber) |
| 18 | Aerosol exit (mouthpiece) |
| 19 | Liquid dosage device |
| 20 | Receptacle |

1. Compressed air inhaler for pulmonary application of a liposomal powder aerosol, entailing a receptacle for a watery liposome dispersion, in which the liposomes are dispersed in water which is connected via a liquid dosing device with an atomiser nozzle and with a drying unit such as an atomisation chamber for spray drying of the liposomes, to which drying unit an exit such as a mouthpiece is connected, wherein the atomiser nozzle has separate supply channels for the compressed air and the liposome dispersion, wherein
the atomiser nozzle comprises a nozzle body with a central cavity, into which an injection body is inserted concentrically, the latter having a central bore for supply of the liposome dispersion spaced adjustably from a release opening of the nozzle body, having a ring-shaped bore with release openings into the cavity of the nozzle body and wherein one of the sides of the nozzle body contains a supply channel for the compressed air parallel to the walls impacting on a bore parallel thereto, the latter connected with the ring-shaped bore of the injection body (2).

2. Compressed air inhaler according to claim 1, wherein
the drying unit has semi-permeable membranes provided, if need be, with a drying agent on the outside.

3. Compressed air inhaler according to claim 1, wherein
a virtual impactor is integrated, by which the drops generated are separated from the atomising air before entering the drying unit.

4. Compressed air inhaler according to claim 1, wherein
the supply of compressed air and the supply of liquid can be adjusted separate from one another.

5-26. (canceled)

27. A method for treating a broncho-pulmonary condition of the respiratory tract in a mammal by administering by inhalation a liposomal dry powder aerosol using the compressed inhaler according to claim 1.

28. The method according to claim 27, wherein the broncho-pulmonary condition is a condition of the trachea and the large bronchial tubes and the aerosol has a particle size of from 5.0 to 10.0 μm.

29. The method according to claim 27, wherein the broncho-pulmonary condition is a condition of the trachea and the large bronchial tubes and the aerosol has a particle size of from 3.0 to 5.0 μm.

30. The method according to claim 27, wherein the broncho-pulmonary condition is a condition of the small bronchial tubes and the aerosol has a particle size of from 0.5 to 3.0 μm.

31. The method according to claim 27, wherein the broncho-pulmonary condition is a condition of the bronchioles and the alveoli and the aerosol has a particle size of from 0.5 to 3.0 μm.

32. A method for manufacturing a liposomal dry powder aerosol by atomizing and drying under pressure an aqueous liposome solution in an atomization and drying chamber using the compressed inhaler according to claim 1.

33. The method according to claim 32, wherein the aerosol has a particle size of from 0.5 to 10.0 μm

34. The method according to claim 32, wherein the aerosol has a particle size of from 5.0 to 10.0 μm.

35. The method according to claim 32, wherein the aerosol has a particle size of from 3.0 to 5.0 μm.

36. The method according to claim 32, wherein the aerosol has a particle size of from 0.5 to 3.0 μm.

37. A powder aerosol produced with the air inhaler according to claim 1.

38. The aerosol according to claim 37, wherein the aerosol has a particle size of from 0.5 to 10.0 μm

39. The aerosol according to claim 37, wherein the aerosol has a particle size of from 5.0 to 10.0 μm.

40. The aerosol according to claim 37, wherein the aerosol has a particle size of from 3.0 to 5.0 μm.

41. The aerosol according to claim 37, wherein the aerosol has a particle size of from 0.5 to 3.0 μm.

42. The air inhaler according to claim 1, wherein the drying unit has semi-permeable membranes with a drying agent on the outside.
